(19) United States
(12) Patent Application Publication
(10) Pub. No.: US 2022/0061920 A1
(43) Pub. Date: Mar. 3, 2022

(54) SYSTEMS AND METHODS FOR MEASURING THE APPOSITION AND COVERAGE STATUS OF CORONARY STENTS

(71) Applicant: Dyad Medical, Inc., Allston, MA (US)

(72) Inventors: John LONG, Shrewsbury, MA (US); Ronny SHALEV, Brookline, MA (US); Soumya Mohanty, Boston, MA (US)

(21) Appl. No.: 17/002,360

(22) Filed: Aug. 25, 2020

(51) Int. Cl. A61B 34/20; A61B 5/00; A61F 2/95; A61B 5/02; G06T 7/73; G06T 7/12

(52) U.S. Cl. CPC A61B 34/20; A61B 5/0066; A61B 5/0084; A61F 2/95; A61B 5/7264; G06T 2207/30101; G06T 7/73; G06T 7/12; A61B 2034/2065; A61B 2576/02; G06T 2207/10101; A61B 5/02007

(57) ABSTRACT

A method and system for detecting coverage status and position of coronary stents in blood vessels by processing intracoronary optical coherence tomography (IVOCT) pullback data performed by software executed on a computer. One example method includes inputting IVOCT pullback data from an imaging device, classifying every image of the IVOCT pullback data into two groups with a binary classification module based on the presence of stent struts in the images, predicting lumen border coordinates from segmentation of every image of the IVOCT pullback data with a lumen segmentation module, identifying objects of interest in every image of the IVOCT pullback data with a stent detection module, and determining the coverage status and position of the coronary stents in blood vessels with an automated analysis application analyzing an output from the binary classification module, an output from the lumen segmentation module, and an output from the stent detection module.

### TECHNICAL FIELD

This specification describes examples of stent and plaque detection and analysis using intravascular optical Coherence Tomography (IVOCT).

### BACKGROUND

Coronary artery disease (CAD) is one of the most common forms of heart disease, which is the leading cause of death in developed countries. To treat CAD stents are placed in the coronary arteries by the means of percutaneous coronary intervention (PCI) procedure. A stent is a tube-like structure made up of a wired mesh designed to be placed in a blood vessel. Its primary purpose is to keep the vessel open. Various stent types have been designed to improve the efficacy of stent treatment. Extensive preclinical and clinical studies are needed to evaluate these newly developed stent designs and perform pre and post deployment evaluations. The drug eluting stent (DES) is the most common type of stent in use today. DES, among types of stents, has been associated with late acquired stent malapposition. A newly deployed stent is generally close to the lumen boundary without any tissue coverage and with time is covered by a thin layer of tissue. However, acute malapposition may occur or the stent may block the blood flow. Hence, detecting the position of stent struts is important for stent placement evaluation and follow-ups.

With superior resolution and imaging speed, intravascular OCT (IVOCT) has been used in-vivo assessment of vessel healing after stent implantation. Low expansion index and a small number of stent struts with tissue coverage may be used as potential biomarkers for late stent thrombosis (LST), an extreme clinical condition with high mortality rate. The percentage of covered stent struts assessed by IVOCT has become an important metric for evaluating stent viability. Recent studies have showed that, with similar percentage of covered struts, a cluster of uncovered struts increases the risk of LST compared to scattered distribution of uncovered struts.

Currently, IVOCT image analysis is primarily done manually, where frames are analyzed in a pre-set increment yet still time consuming, error-prone and biased. IVOCT requires extensive specialized training, which limits the number of physicians qualified to use IVOCT. Interpretation of IVOCT images is also difficult and can be time consuming. Furthermore, during a typical PCI, a single pullback may create over five hundred images, overloading the physician with data during an already stressful intervention. In addition, inter- and intra-observer variability is inevitable in manual analysis. Therefore, there is need for a computerized and automated stent analysis solution that can address these problems by reducing time and labor costs and by increasing reliability and reproducibility of stent analysis results.

### SUMMARY

In a first embodiment, method of detecting coverage status and position of coronary stents in blood vessels by processing intracoronary optical coherence tomography (IVOCT) pullback data performed by software executed on a computer is provided. The method includes inputting IVOCT pullback data from an imaging device, classifying every image of the IVOCT pullback data into two groups with a binary classification module where a first group and a second group are determined by a presence of stent struts in images of the IVOCT pullback data, predicting lumen border coordinates from segmentation of every image of the IVOCT pullback data with a lumen segmentation module, identifying objects of interest in every image of the IVOCT pullback data with a stent detection module, and determining the coverage status and position of the coronary stents in blood vessels with an automated analysis application analyzing an output from the binary classification module, an output from the lumen segmentation module, and an output from the stent detection module

In a second embodiment, a system for detecting coverage status and position of coronary stents in blood vessels by processing intracoronary optical coherence tomography (IVOCT) pullback data performed by software executed on a computer is provided. The system includes an IVOCT device for acquiring IVOCT pullback data from a patient, a computer for processing the IVOCT pullback data with a method for detecting coverage status and position of coronary stents in blood vessels where the method includes inputting IVOCT pullback data from an imaging device, classifying every image of the IVOCT pullback data into two groups with a binary classification module where a first group and a second group are determined by a presence of stent struts in images of the IVOCT pullback data, predicting lumen border coordinates from segmentation of every image of the IVOCT pullback data with a lumen segmentation module, identifying objects of interest in every image of the IVOCT pullback data with a stent detection module, identifying objects of interest in every image of the IVOCT pullback data with a stent detection module, and determining the coverage status and position of the coronary stents in blood vessels with an automated analysis application analyzing an output from the binary classification module, an output from the lumen segmentation module, and an output from the stent detection module, and a display screen to display the IVOCT pullback data and the coverage status and position of the coronary stents in blood vessels generated by the method.

DESCRIPTION OF THE DRAWINGS

The accompanying drawings, which are incorporated in and constitute a part of the specification, illustrate various example methods, and other example embodiments of various aspects of the invention. It will be appreciated that the illustrated element boundaries (e.g., boxes, groups of boxes, or other shapes) in the figures represent one example of the boundaries. One of ordinary skill in the art will appreciate that in some examples one element may be designed as multiple elements or that multiple elements may be designed as one element. Furthermore, elements may not be drawn to scale.

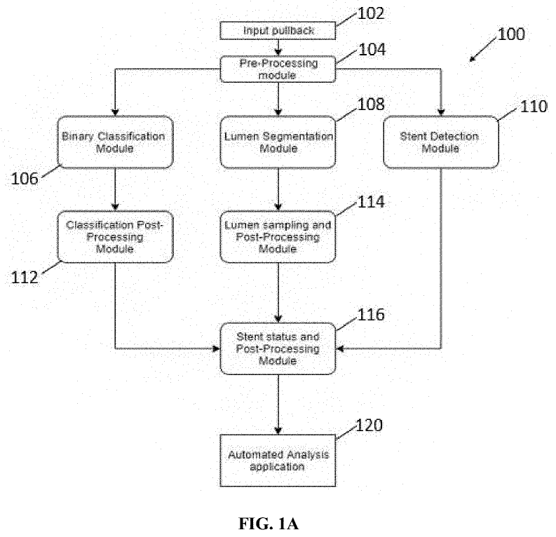

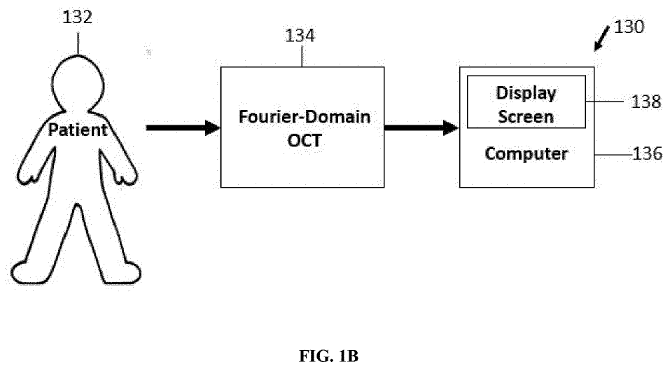

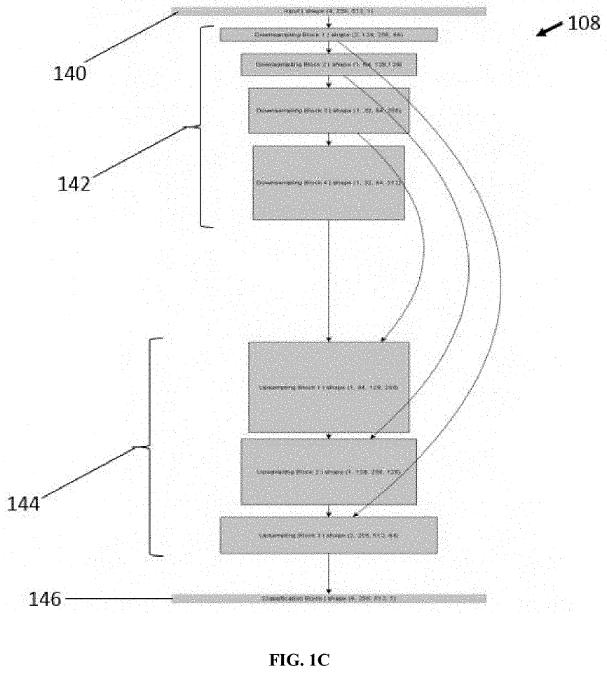

FIG. 1A illustrates a flowchart of an example method for visualization and analysis of IVOCT image pullbacks in accordance with one illustrative embodiment.

FIG. 1B illustrates a schematic of the example method for visualization and analysis of IVOCT image pullbacks in accordance with FIG. 1A.

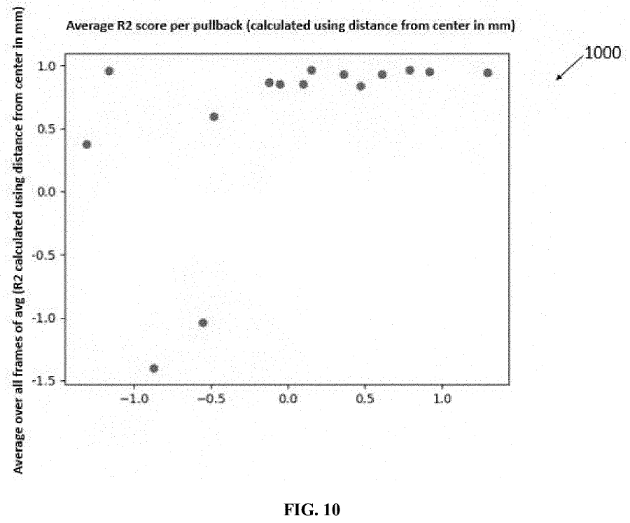

FIG. 10 illustrates a flowchart of a lumen segmentation module of the method for visualization and analysis of IVOCT image pullbacks in accordance with FIG. 1A.

FIG. 2 illustrates an example graph depicting the receiver operating characteristic curve and the true positive rate versus the false positive rate.

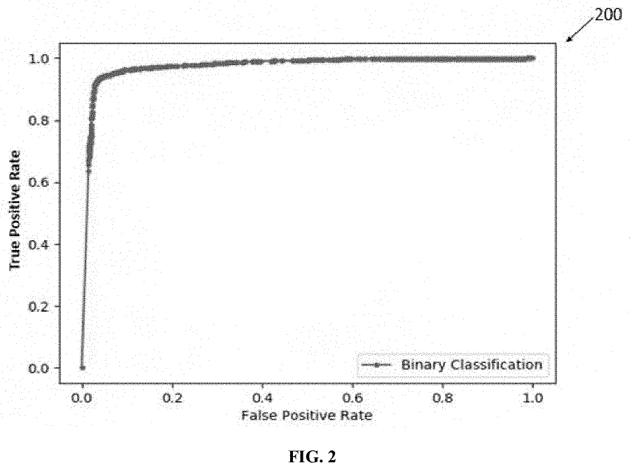

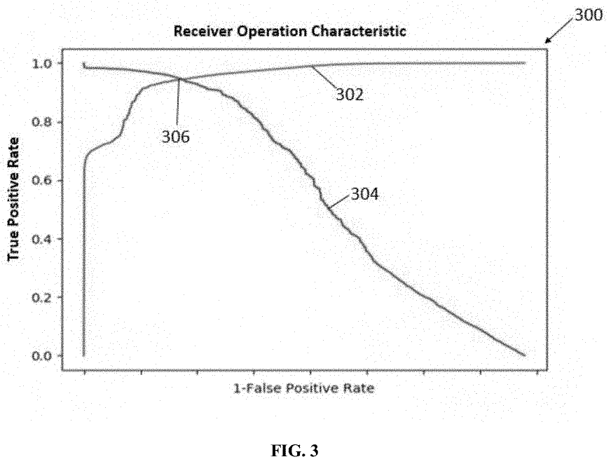

FIG. 3 illustrates an example graph depicting the receiver operating characteristic curve and the true positive rate versus the 1-false positive rate.

FIG. 4 illustrates a confusion matrix for binary classifier along with the accuracy.

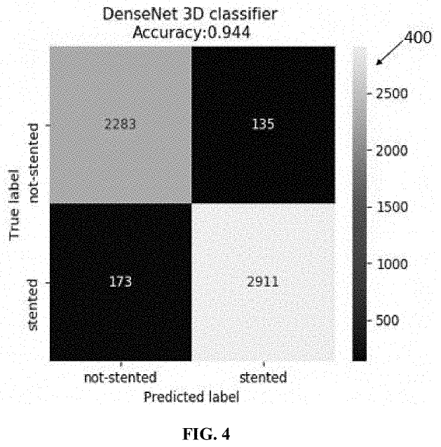

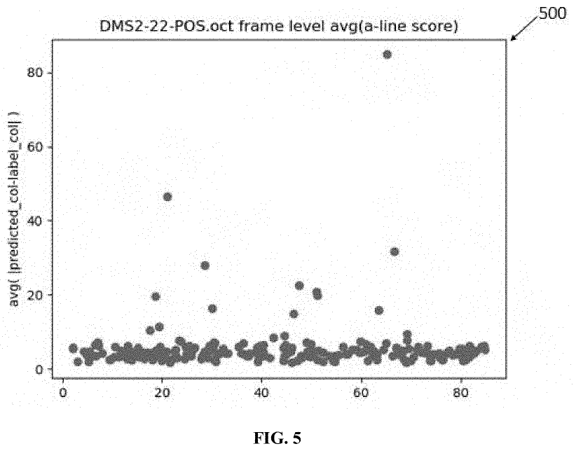

FIG. 5 illustrates an example scatter plot of sum of absolute values of the difference between the predicted coordinate and the label coordinate for all frames of one of the IVOCT image pullbacks used for testing.

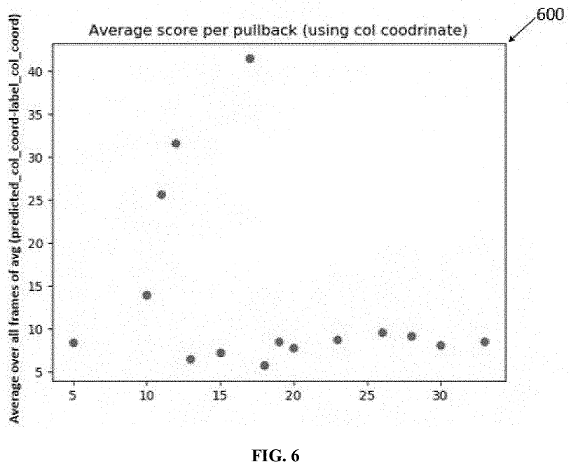

FIG. 6 illustrates an example scatter plot of average of the sum of error term per IVOCT image pullback.

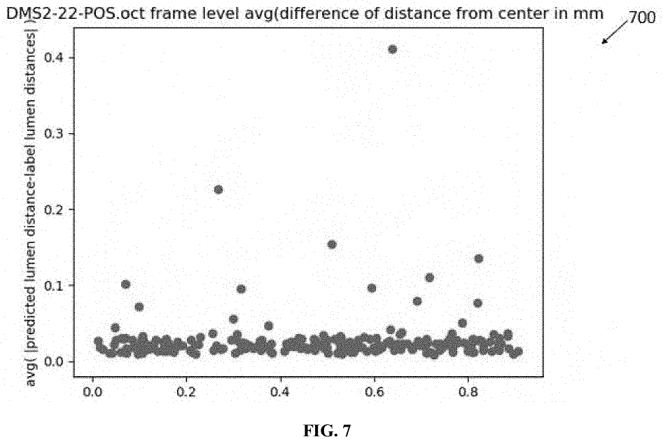

FIG. 7 illustrates an example scatter plot of sum of absolute values of the difference between the predicted distance of the lumen border from the center in mm and the ground truth distance of the lumen from the center (mm) for all frames of one of the IVOCT image pullbacks used for testing

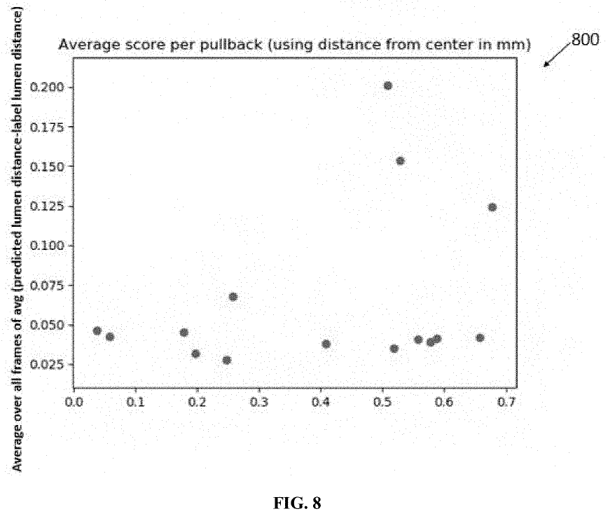

FIG. 8 illustrates an example scatter plot of average of the sum of absolute values of the difference between the predicted distance of the lumen border from the center (mm) and the ground truth distance of the lumen from the center (mm) per IVOCT image pullback.

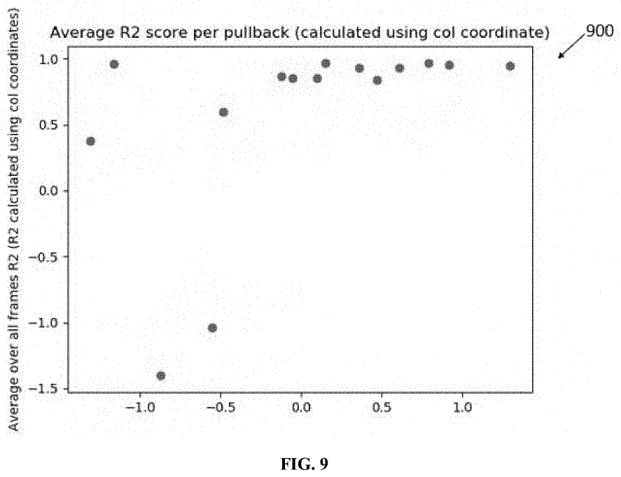

FIG. 9 illustrates an example scatter plot showing average r-squared score for all frames per IVOCT image pullback.

FIG. 10 illustrates an example scatter plot showing average r-squared score calculated using distance of lumen from the center (mm) for all frames per IVOCT image pullback.

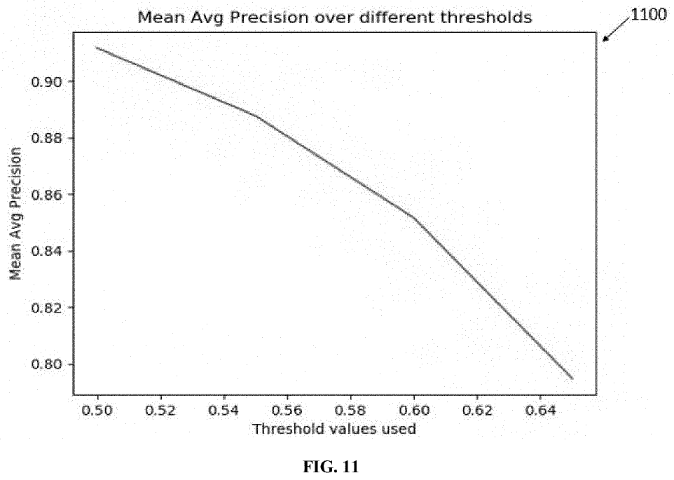

FIG. 11 illustrates an example plot showing mean average precision versus threshold values.

### DETAILED DESCRIPTION

FIGS. 1A and 1B illustrate an exemplary method 100 and system 130 for visualization and analysis of IVOCT image pullbacks in accordance with one illustrative embodiment. The method 100 depicts the overview of an algorithm which automatically segment lumen boundaries of blood vessels, detect stent struts and guide wires. The method 100 also determines the tissue coverage based on borders of lumens of blood vessels and classifies stent struts into specified categories such as apposed, malapposed, covered, and uncovered. The method 100 can be performed by a computer 136 connected to a Fourier-Domain OCT system 134 used to acquire image pullbacks (stacks of images from arteries) from a patient 132. The computer includes a display screen 138 to display the pullbacks, as well as a stent analysis computed by the method 100. Preferably, the method 100 will detect the position of the stent struts for stent placement evaluation and follow-ups for display to a caregiver at the point of care, or remotely.

In one embodiment, the automated algorithm of the computerized method 100 of FIG. 1A utilizes an input pullback 102 from real clinical exams conducted with the Fourier-Domain OCT system 134 on patients 132. Each clinical examination yields data for an input pullback 102, which is a collection of images analyzing a section of a blood vessel. In one embodiment, the Fourier-Domain OCT system 134 was equipped with a tunable laser light source sweeping from 1250 nm to1370 nm, providing 15μm resolution along an A-line. The input pullback 102 from the Fourier-Domain OCT 134 was acquired at speed of 20 mm/sec over a distance of 54.2 mm with a 200 μm interval between frames, giving 217-375 total images (also referred to as frames). These frames were collected in the r-theta format. Each polar-coordinate (r, θ) image consisted of at least 504 A-lines and at least 900 pixels along the A-line, and 16 bits of gray-scale data. In the embodiment described above, the computerized method 100 was trained with a dataset comprising 1744 pullbacks, with a total of 624,078 frames to locate regions for stent placement and stent analysis after placement.

The computerized method 100 of FIG. 1A includes a pre-processing module 104. The images in the input pullback 102 are usually 16-bit raw images with no adjustments in color or contrast. In one embodiment, the images in the input pullback 102 are converted from 16-bit one channel or three channel images to 8-bit single channel images. In the pre-processing module 104, the images in the input pullback 102 are automatically resized, window-leveled and normalized by dividing the pixel values by the highest pixel value found in the image.

The computerized method 100 of FIG. 1A further includes a binary classification module 106 and a classification post-processing module 112. The binary classification module 106 confirms the presence of a stent in each image output following the pre-processing module 104. The binary classification module 106 was created based on a three-dimensional variation of a commonly known dense convolutional network (DNC) called DenseNet. In one embodiment, the binary classification module 106 has software architecture made up of 61 layers and utilizes ensemble learning, to generate accurate output to be used in the classification post-processing module 112. The binary classification module 106 of computerized method 100 of FIG. 1A can process more than one image at a time. In the above stated embodiment, the binary classification module 106 was made to process four images simultaneously. The output from each image is averaged and used as input for the classification post-processing module 112. The classification post-processing module 112 is used to generate an output denoting whether the input pullback 102 contains a stent, and also a starting and ending location of the stent in each of the images.

The computerized method 100 of FIG. 1A also includes a lumen segmentation module 108 and a lumen sampling and post-processing module 114. The lumen segmentation module 108 predicts the coordinates of the lumen border on each frame in each image output following the pre-processing module 104. The lumen segmentation module 108 was created with a three-dimensional variation of a software architecture based on U-Net.

In one embodiment, the software architecture of the lumen segmentation module 108 includes an input 140 and three major components, a downsampling block 142, an upsampling block 144 and a classification layer 146 as shown in FIG. 10. The downsampling block 142 and an upsampling block 144 each comprises multiple sub-blocks respectively. Each sub-block of the downsampling block 142 is parametrized with one nb_filters parameter specifying a number of filters used in convolution layers and a last layer sub-block of the downsampling block 142. An ordering of the convolution layers in the downsampling block 142 is a convolutional layer of nb_filters with relu activation function. This is followed by another convolutional layer with the same parameters. Finally, maxpooling is used to reduce the size of the input to the layer. Dropout is then applied to the pooled output.

Each sub-block of the upsampling block 144 is used to expand feature maps and gradually position each feature to the location of the feature in the original image. The upsampling block 144 takes in two layers and a number specifying the number of filters as arguments. First, the immediately preceding layer is upsampled through nearest neighbor interpolation to increase image size and put through a convolutional layer. Then, the same layer is stacked on top of the previous feature map from the contracting path with the same image dimensions. This is followed by two additional convolutions.

The classification layer 146 is a convolutional layer with a 1×1 kernel and exactly one filter. This results in a value at each position of the image representing the probability of lumen border. In one embodiment, there are 4 downsampling layers that perform convolution. The first two downsampling block 142 decreases the image size, decreasing the length and width and depth by a factor of 2. The third downsampling layer sub-block decreases only the height and width by a factor of 2, leaving the depth unchanged. The fourth downsampling layer sub-block performs no pooling. Each downsampling layer sub-block increases the number of filters by a factor of 2 from the previous downsampling layer sub-block. In the above mentioned embodiment, 3 upsampling layers sub-blocks increase the image size and each subsequent upsampling layer sub-block has half as many filters as the previous.

The output from each image from the lumen segmentation module 108 is averaged to yield a good initial prediction of the lumen border and used as input for the lumen sampling and post-processing module 114. The lumen sampling and post-processing module 114 is used to generate an output of the model by randomly sampling parts of the predicted border and then generating a spline along the sampled border, to generate a final lumen segmentation output.

The computerized method 100 of FIG. 1A further comprises a stent detection module 110. The stent detection module 110 is used for the detection of stent struts and guide wires in a given input pullback 102 for each image output following the pre-processing module 104. The stent detection module 110 was created as variant of the Faster-RCNN model. The stent detection module 110 has a software architecture containing a 101 layer ResNet and pre-trained on imageNet for object classification. In one embodiment, the stent detection module 110 was trained using 53 input pullbacks 102 (14,164 images) to provide an output.

A stent status and post-processing module 116 analyzes the output of the stent detection module 110 detection module 110, the output of the lumen sampling and post-processing module 114, and the classification and post-processing 112 module to obtain candidate stent struts by removing images that were incorrectly labelled as comprising stent struts but actually classified as non-stented by the binary classification module 106. These candidate struts are further processed using a shadow matching algorithm presented in the stent status and post-processing module 116 to determine the center of each strut found. The output of the stent status and post-processing module 116 is used as input for an automatic analysis application 120. The automatic analysis application 120 further calculates the distance between the lumen border and center of the stent strut. In one embodiment, the automatic analysis application 120 is used to determine an apposition status based apposition status and coverage status of the each candidate strut detected by comparing the thickness of the strut and the distance between the lumen border and center of the stent strut.

### Results

### Binary Classification Module

FIG. 2 illustrates an exemplary graph 200 depicting the receiver operating characteristic (ROC) curve of the true positive rate versus the false positive rate. In one embodiment, the three-dimensional DenseNet model based binary classification module 106 was tested on a held-out set of 20 input pullbacks 102 (5502 images) to identify the presence of stent struts. 3084 images (56%) were found to have stent struts and 2418 images (43%) were found to not have stent struts. To demonstrate the validity of the results and diagnostic ability of the binary classification module 106, an area under the ROC of curve of the true positive rate versus the false positive rate were calculated as the area under the ROC curve represents the ability of the binary classification module 106 to distinguish between the images with stent struts and the image without stent struts. In one embodiment, the area under the ROC curve was 0.975052 (97%), a strong indication of the reliability of the binary classification module 106 to differentiate between an image with stent struts and an image without stent struts.

FIG. 3 illustrates an exemplary graph 300 depicting the ROC curve of the true positive rate versus the 1-false positive rate. The exemplary graph 300 includes a true positive rate curve 302 and a 1-false positive rate curve 304. An optimal threshold 306 is determined by the intersection of true positive rate curve 302 and the 1-false positive rate curve 304 and subtracting a value of 1-false positive rate from a true positive rate. As depicted in the exemplary graph 300, in one embodiment, the optical threshold 306 is identified as having a value of 0.115 where the true positive rate is 0.9442 (or 94%) and the false positive rate is 0.0558 (or 5%). The optimal threshold 306 is used as a probability threshold where any prediction with a probability greater than the optimal threshold 306 as containing stents and any prediction with a probability below than the optimal threshold 306 as containing stents.

FIG. 4 illustrates an exemplary confusion matrix 400 for binary classifier along with the accuracy based on the optimal threshold 306 determined in FIG. 3 to classify the images as containing stents or not containing stents. The exemplary confusion matrix 400 is used to derive an overall accuracy of the binary classification module 106 by calculating a sensitivity and a specificity the binary classification module 106. The sensitivity and specificity are defined as follows:

$$Sensitivity = \frac{True Positives}{True Positives + False Negatives}$$

$$Specificity = \frac{True Negatives}{False Positives + True Negatives}$$

In one embodiment, the binary classification module 106 provided a prediction of 2283 true negatives (images without stents classified as non-stented), 135 false positives (images without stents classified as stented), 2911 true positives (image with stents classified as stented), and 173 false negatives (images with stents classified as non-stented). The sensitivity was calculated to be 0.94 (94%) and the specificity was calculated to be 0.9441 (94%), to derive an overall accuracy of 94%.

The exemplary confusion matrix 400 of FIG. 4 is further used in the classification post-processing module 112 by taking a sequence of images and building image groups. Building the image groups is used as a filter to identify standalone images to further remove false positives and accurately predict the accurate location of the stent in terms of starting and ending images where the stent exists. An accuracy after post processing by the classification post-processing module 112 is determined to be +/−1frame in terms of whether the given input pullback 102 contains a stent or not.

### Lumen Segmentation Module

FIG. 5 illustrates an exemplary scatter plot 500 of sum of absolute values of the difference between the predicted coordinate and the label coordinate for all frames of one of the IVOCT image input pullbacks 102 used for testing in the lumen segmentation module 108. In one embodiment, the lumen segmentation module 108 was tested with 15 input pullbacks 102 containing 3416 images. Out of the 453,600 pixels per image, the lumen segmentation module 108 only segments one pixel per row corresponding to a height of the image. To evaluate the goodness of the segmentation, a custom sum of absolute difference metric was computed. The sum of absolute differences metric, in terms of column coordinate predicted is:

$${\sum }_{i = 1}^{H} \vert Pc - c \vert$$

Where, H denotes the number of a-lines in the image. a denotes the column coordinate predicted by the model and GT_{c} denotes the ground truth column coordinate.

The lumen segmentation module 108 used the custom sum of absolute difference metric to predict the column coordinates for the lumen border in r-theta for each A-line of each input pullback 102 and displayed the results in the form of the exemplary scatter plot 500 of FIG. 5. In the embodiment used to depict the exemplary scatter plot 500 of FIG. 5, the custom sum of absolute difference metric was low apart from a few images, validating the reliability of the lumen segmentation module 108 to predict the lumen border.

FIG. 6 illustrates an exemplary scatter plot 600 of average of the sum of error term per IVOCT image input pullback 102. A score based on the custom sum of absolute difference metric on for all the images in input pullback 102 calculated and averaged. In one embodiment, the average score was calculated each of the 15 input pullbacks 102 and displayed as the exemplary scatter plot 600 of FIG. 6. The exemplary scatter plot 600 of FIG. 6 illustrates a low average error we get when comparing the column coordinates on a pullback level. As the averages of scores between the predicted coordinate and the label coordinate for all frames in a pullback are low, it can deduced that there was only a minor difference between the lumen border detected and the actual ground truth border.

FIG. 7 illustrates an exemplary scatter plot 700 of sum of absolute values of the difference between the predicted distance of the lumen border from the center (mm) and the actual ground truth distance of the lumen from the center (mm) for all frames of one of the IVOCT image pullbacks used for testing in the lumen segmentation module 108. A custom sum of absolute difference metric in terms of distance of the lumen from the center in millimeters, instead of column coordinates as described in the above paragraphs, was computed from the images in the input pullbacks 102 by the following equation:

$${\sum }_{i = 1}^{H} \vert {Pd}_{mm} - {GTD}_{mm} \vert$$

Where, H denotes the number of a-lines in the image. PDₘₘ denotes the predicted distance from center to lumen border in millimeters. And GTDₘₘ denotes the ground truth distance from center to lumen border in millimeters.

In one embodiment, the custom sum of absolute difference metric in terms of distance of the lumen from the center in millimeters computed and displayed as the exemplary scatter plot 700 of FIG. 7. The scatter plot 700 shows that the error term for most frames in the input pullback 102 is minimal and the difference between the predicted distance is on an average 0.1 mm away from the ground truth distance of lumen from the center.

FIG. 8 illustrates an exemplary scatter plot 800 of average of the sum of absolute values of the difference between the predicted distance of the lumen border from the center (mm) and the ground truth distance of the lumen from the center (mm) per IVOCT image pullback. The custom sum of absolute difference metric in terms of distance of the lumen from the center in millimeters is computed for all the image in each input pullback 102 and averaged to produce an average score per input pullback 102. In one embodiment, the average score of each pullback used as input pullbacks 102 are computed and displayed as the exemplary scatter plot 800 of FIG. 8. From the exemplary scatter plot 800 of FIG. 8, it can be deduced that the average of the sum of absolute error scores both in terms of column coordinates and distance from center in millimeters is very low for the majority of the input pullbacks 102. It can also be observe that when using the distance of lumen from the center in millimeters the largest average error term computed is 0.2 mm, a minor error validating the accuracy of the lumen segmentation module 108.

FIGS. 9 and 10 illustrate exemplary scatter plot 900 and exemplary scatter plot 1000 showing average r-squared score for all frames based on the coordinates and distance of lumen from the center (mm) for all frames per IVOCT image pullback respectively. The goodness of the segmentation of the lumen segmentation module 108 is also evaluated by an R-squared test metric. The R-squared test metric is used to compute an R-squared score for each image in every input pullback 102 after which the R-squared scores were averaged to deduce an average R-squared score for every input pullback 102 and depicted as exemplary scatter plot 900 and exemplary scatter plot 1000. In one embodiment, the exemplary scatter plots 900 and 1000 show that the R-squared value was close to 1 in both instances, denoting the accuracy of the lumen segmentation model 102.

### Stent Detection Module

FIG. 11 illustrates an exemplary plot 1100 showing mean average precision versus threshold values computed from the stent detection module 110. The stent detection module 110 is used to compute bounding boxes from the input pullbacks 102 and compare with ground truth boxes manually computed. An intersection over union (IOU) is calculated as means of comparison between the computed bounding boxes and the ground truth boxes to evaluate the stent detection module 110. In one embodiment, the stent detection module 110 was trained using 53 input pullbacks 102 (14,164 images). Out of the entire training set, 11,332 images (80%) were used for training and 2832 images (20%) were used for validation. A separate held out test set of 605 images was used to test the performance of the stent detection module 110.

In one embodiment, the stent detection module 110, which can detect stents struts and guide wires, presents with an average precision for guide wire detection of 98.46% and average precision for strut detection is 83.87%. This presents a mean average precision (mAP) value of 91.17% validating the accuracy of the stent detection module 110. The stent detection module 110 also comprises a shadow matching algorithm which is then applied to regions of interest identified and used to detect locations of centers of struts. The locations of these centers in conjunction with the output of the lumen segmentation model 108 are used for classifying these struts into various categories like apposed, malapposed, covered and uncovered.

References to “one embodiment”, “an embodiment”, “one example”, and “an example” indicate that the embodiment(s) or example(s) so described may include a particular feature, structure, characteristic, property, element, or limitation, but that not every embodiment or example necessarily includes that particular feature, structure, characteristic, property, element or limitation. Furthermore, repeated use of the phrase “in one embodiment” does not necessarily refer to the same embodiment, though it may.

To the extent that the term “includes” or “including” is employed in the detailed description or the claims, it is intended to be inclusive in a manner similar to the term “comprising” as that term is interpreted when employed as a transitional word in a claim.

Throughout this specification and the claims that follow, unless the context requires otherwise, the words ‘comprise’ and ‘include’ and variations such as ‘comprising’ and ‘including’ will be understood to be terms of inclusion and not exclusion. For example, when such terms are used to refer to a stated integer or group of integers, such terms do not imply the exclusion of any other integer or group of integers.

To the extent that the term “or” is employed in the detailed description or claims (e.g., A or B) it is intended to mean “A or B or both”. When the applicants intend to indicate “only A or B but not both” then the term “only A or B but not both” will be employed. Thus, use of the term “or” herein is the inclusive, and not the exclusive use. See, Bryan A. Garner, A Dictionary of Modern Legal Usage 624 (2d. Ed. 1995).

While example systems, methods, and other embodiments have been illustrated by describing examples, and while the examples have been described in considerable detail, it is not the intention of the applicants to restrict or in any way limit the scope of the appended claims to such detail. It is, of course, not possible to describe every conceivable combination of components or methodologies for purposes of describing the systems, methods, and other embodiments described herein. Therefore, the invention is not limited to the specific details, the representative apparatus, and illustrative examples shown and described. Thus, this application is intended to embrace alterations, modifications, and variations that fall within the scope of the appended claims.

What is claimed is:

1. A method of detecting coverage status and position of coronary stents in blood vessels by processing intracoronary optical coherence tomography (IVOCT) pullback data performed by software executed on a computer, the method comprising:
inputting IVOCT pullback data from an imaging device;
classifying every image of the IVOCT pullback data into two groups with a binary classification module, wherein a first group and a second group are determined by a presence of stent struts in images of the IVOCT pullback data;
predicting lumen border coordinates from segmentation of every image of the IVOCT pullback data with a lumen segmentation module;
identifying objects of interest in every image of the IVOCT pullback data with a stent detection module; and
determining the coverage status and position of the coronary stents in blood vessels with an automated analysis application analyzing an output from the binary classification module, an output from the lumen segmentation module, and an output from the stent detection module.

2. The method according to claim 1, further comprising a lumen sampling and post-processing module for randomly sampling parts of predicted lumen border coordinates from the lumen segmentation module, where the predicted lumen border coordinates is used to generate a spline along the predicted lumen border to generate a final lumen segmentation output.

3. The method of according to claim 1, further comprising a classification post-processing module for analyzing the output of the binary classification module to determine a starting and ending locations of stents in each images of the IVOCT pullback data.

4. The method according to claim 1, further including a stent status and post-processing module to analyze the output from the binary classification module, the output from the lumen segmentation module, and the output from the stent detection module to determine the center of each of the stent struts detected.

5. The method according to claim 1, further comprising a pre-processing module to convert IVOCT pullback data from the imaging device from 16-bit single channel or three channel images to 8-bit single channel images.

6. The method according to claim 1, wherein the binary classification module can process more than image from the IVOCT pullback data from the imaging device simultaneously.

7. The method according to claim 6, wherein the binary classification module can process four images from the IVOCT pullback data from the imaging device simultaneously.

8. The method according to claim 4, further including calculating a distance between the lumen border and the center of each stent strut.

9. The method according to claim 8, further determining the position of the coronary stents by comparing the distance between the lumen border and the center of each stent strut and the thickness of the coronary strut.

10. A system for detecting coverage status and position of coronary stents in blood vessels by processing intracoronary optical coherence tomography (IVOCT) pullback data performed by software executed on a computer, the system comprising:
an IVOCT device for acquiring IVOCT pullback data from a patient;
a computer for processing the IVOCT pullback data with a method for detecting coverage status and position of coronary stents in blood vessels, the method comprising:
inputting IVOCT pullback data from an imaging device;
classifying every image of the IVOCT pullback data into two groups with a binary classification module, wherein a first group and a second group are determined by a presence of stent struts in images of the IVOCT pullback data;
predicting lumen border coordinates from segmentation of every image of the IVOCT pullback data with a lumen segmentation module;
identifying objects of interest in every image of the IVOCT pullback data with a stent detection module; and
determining the coverage status and position of the coronary stents in blood vessels with an automated analysis application analyzing an output from the binary classification module, an output from the lumen segmentation module, and an output from the stent detection module; and
a display screen to display the IVOCT pullback data and the coverage status and position of the coronary stents in blood vessels generated by the method.
